(19) United States
(12) Patent Application Publication
(10) Pub. No.: US 2007/0142739 A1
(43) Pub. Date: Jun. 21, 2007

(54) Sensoric method for evaluating breath

(76) Inventors: Steffen Sonnenberg, Holzminden (DE); Anja Finke, Holzminden (DE); Arnold Machinek, Holzminden (DE); Bianca Piper, Hoxter-Stahle (DE); Jurgen Rabenhorst, Hoxter (DE)

Correspondence Address: ROYLANCE, ABRAMS, BERDO & GOODMAN, L.L.P., 1300 19TH STREET, N.W., SUITE 600, WASHINGTON,, DC, 20036, US

(21) Appl. No.: 10/564,158

(22) PCT Filed: Jun. 24, 2004
(86) PCT No.: PCT/EP04/51229

§ 371 Date: Apr. 26, 2006

(30) Foreign Application Priority Data
Jul. 11, 2003 (DE) 103 31 379.6

(51) Int. Cl. A61B 5/08

(52) U.S. Cl. 600/532

(57) ABSTRACT

The invention relates to a novel method of evaluating the breath of a test subject. A breath sample from a test subject is collected in a container. The microbial load of the breath sample collected in the container is reduced. The breath sample collected in the container is evaluated by a tester.

The present invention relates to a sensory method of evaluating breath. The method is particularly suitable for the anonymous testing, free from microbiological load, of the effective and long-lasting removal of mouth odour.

A considerable problem in oral hygiene is bad breath, also known as mouth odour, oral malodour or halitosis. This odour is caused by numerous volatile compounds, such as e.g. hydrogen sulfide, methylmercaptan, methyl sulfide, skatole and indole. These compounds are formed by the decomposition of food remains and dead mucous membrane cells by many different microorganisms, such as gram-positive and gram-negative microorganisms and/or protozoa. Anaerobic gram-negative bacteria are mentioned as an example (Bad Breath—A multidisciplinary Approach. Eds: D. van Steenberghe, M. Rosenberg, Leuven University Press, Leuven 1996; 111-121).

To improve oral hygiene, medicinal dental treatments and oral care products, such as e.g. chewing gum, toothpaste or mouthwashes, are used. The oral care products are intended to permit long-lasting, odour-free breath. In addition, the impression of long-lasting freshness in the breath can be achieved by means of various flavourings in the oral care products.

The organoleptic evaluation of human breath is performed inter alia by one or more test subjects breathing directly on to one or more testers (Rosenberg M and McCulloch A G, Measurement of oral malodor: Current methods and future prospects. J Periodontol 63:776-82, 1992). Organoleptic evaluations are also performed by commercial institutes (e.g. Hilltop www.hill-top.com/capabilites/Oral%20Care.html or www.hill-top.com/Capabilities/Oral Care). In general, tester groups of about two to four testers assess the breath directly from the mouth of test subjects. Approximately 30 test subjects are required to achieve a reliable statement. A disadvantage of the known method is that the tester comes into direct contact with the test subject. As a result, the odour evaluation of the breath can be influenced by other factors. Furthermore, the direct and repeated comparison of two or more test subjects is impossible.

Alternatively, the tester and test subject are separated from one another by a thin wall or a cloth with a small hole, through which the test subject breathes. However, in both methods the test subject and the tester still come into contact sufficiently for a psychic load to arise. Only a few test subjects, but in particular only a very few testers, can be subjected to this load without being influenced. This means that the quality of the evaluation and the number of tests that can be conducted is limited because of the small number of qualified testers available.

In addition, when breathing directly on to the tester, numerous microorganisms can be transferred from the test subject to the tester by means of aerosols. Particularly in the case of pathological mouth odour, as caused e.g. by halitosis, these microorganisms can result in harm to the tester's health.

Products for the assessment of breath are known in which a test subject blows his breath into reagent solutions (cherrystone corporation, Diagnostics for Healthcare, PA, 19087-5835, USA, inter alia). These reagent solutions react with sulfurous volatile compounds from the breath to give a colour reaction. In this method, only a very rough estimate of the bad breath is possible. In addition, other malodorous compounds, such as skatole and indole, are not detected. It is not possible to test the freshness effect of oral care products by this method.

In addition, instruments are known for the electronic assessment of breath, such as the “Fresh Kiss” (www.pro-omnia.de or www.pro-omnia.de/docs/pd999814474.htm) or the “Halimeter” (www.halithose.de/halimtr.htm or www.halimeter.com). In both instruments, a test subject breathes on to an electronic odour sensor. The sensor determines the concentration of volatile sulfur compounds present in the gas phase. The data thus obtained do not permit any quantification of the strength of mouth odour, however, and so a doctor has to take into account other test methods, such as e.g. bacterial cultures, sulfide detection methods and organoleptic methods (sniffing), for the final diagnosis. In addition, after the use of a flavoured oral care product, instruments also indicate apparently bad breath because of the volatile flavourings. There is not yet, therefore, any generally usable substitute for an organoleptic assessment of the breath by one or more testers.

From WO 97/00444, a device and a method are known for evaluating the condition of an economically useful animal, especially a ruminant. A sample of the animal's breath is collected in a pouch and then analysed by an electronic odour sensor. The sensor should, in particular, be able to detect antibodies in mucous particles that are carried over in the breath. It is a disadvantage of this method and the corresponding device that no assessment of the strength and nature of the odour is possible.

The object of the present invention was therefore to improve the conventional evaluation methods in such a way that greater reproducibility of the evaluation results is made possible. The method should additionally reduce the psychic loads associated with the evaluation of human breath for the test subject and the tester and also, as far as possible, reduce the risk of infection between the test subject and the tester.

The object is achieved by a method of evaluating the breath of a test subject, comprising the following steps:
a) collection of a breath sample from the test subject in a container
b) reduction of the microbial load of the breath sample contained in the container, and then
c) evaluation by a tester of the breath sample collected in the container.

The use of a container for collecting a breath sample from a test subject for evaluation of the breath sample by a tester offers numerous advantages. In particular, the microbial load of the breath to be evaluated is reduced surprisingly strongly and rapidly by storing the breath sample in a container. The comparison of the number of microorganisms in the two procedures can be detected by direct or indirect breathing from the container by microbiological test systems, such as a microbial air sampler (e.g. an MD 8 airscan system, Sartorius, D-Gottingen). Within the meaning of the present invention, the microbial load is reduced when the microbial load in the gas phase of a breath sample present in the container is smaller than the microbial load in the gas phase of a directly exhaled breath sample.

Despite this change in the breath sample, it has surprisingly been found that the odour of the breath sample, i.e. the strength and nature of its odour, does not noticeably change compared with the odour of breath that has been directly exhaled. It was particularly surprising that the odour of the breath sample does not change within a storage period of half an hour, even when the breath sample is evaluated by a human tester instead of an electronic nose, although the microbial load in the gas phase of the breath sample decreases markedly. The method according to the invention therefore makes it possible, for the first time, for a breath sample to be collected for an evaluation that does not take place until some time after the breath sample has been provided in step a).

Because the test subject and the tester no longer meet directly, the psychic load associated with breath evaluation is also reduced, both for the test subject and for the tester. As a result, the objectivity of the evaluation is increased and the reproducibility of the evaluation results is improved. The new method also results in a marked increase in the acceptance of breath assessment on the part of the testers, so that more testers can be recruited for a breath assessment. Because of the greater number of available and qualified testers and suitable test subjects, the quality of the evaluation increases.

A further advantage of the method according to the invention is that the breath sample can be evaluated several times because of its storage stability. In particular, a breath sample can be evaluated by several testers in step c) and, if necessary, the evaluation can also be repeated. In conventional procedures, the test subject would have had to breathe on a tester again each time for this purpose. The method according to the invention makes it possible, for the first time, to assess the effectiveness of a long-term treatment—e.g. over several weeks. For this purpose, the intensity of the typical mouth odour is assessed at the beginning and end of the treatment, it being possible to compare breath samples taken at the beginning and end of the treatment directly in step c). After taking the samples in step a), the breath samples are preferably stored for no longer than 7 hours before being evaluated in step c). The storage period is particularly preferably no more than 5 hours, especially 10 minutes to 30 minutes.

The method according to the invention makes it possible to make reliable statements as to the effective and long-lasting elimination of mouth odour and the freshness effect of oral care products. Among other things, the method according to the invention is suitable for comparing the effective and long-lasting elimination of mouth odour and the freshness effect of two or more oral care products.

The method according to the invention for the evaluation of breath can be carried out at any time and under widely varying conditions. Thus, for example, the intensity of the typical mouth odour can be evaluated in the morning, after treating the oral cavity with dental care products the previous evening. In particular, the evaluation of the effectiveness of a treatment of the oral cavity for food odours, such as e.g. garlic, onions or other highly flavoured foods, is possible. The method according to the invention is preferably used to assess the success of a medical treatment of the oral cavity, such as e.g. the removal of deposits from teeth, gums, the tongue or the remainder of the oral mucosa, this deposit having been removed e.g. by mechanical or chemical agents.

In step a), the container is preferably filled with breath by the test subject against atmospheric pressure by exhalation. This prevents adulteration of the breath sample. In preliminary tests, it has been shown that, when a breath sample is sucked out of a stream of air being breathed, a significant change occurs in the odour of the breath sample compared with the odour of the breath that has been directly exhaled. On the other hand, the odour of a breath sample of the directly exhaled breath changes even when the test subject has to blow the breath sample into a container instead of only exhaling, thus breathing against more than atmospheric pressure, as when blowing up a balloon.

In preliminary tests, it has proved particularly useful if the container used to collect the breath sample possesses a variable volume, it being particularly advantageous if the container is collapsible. Since such containers contain no air, or only a small quantity of air, before the breath sample is collected, they can easily be filled with a breath sample without diluting it with atmospheric air. Collapsible containers also have the advantage that it is possible to breathe into them with normal breathing pressure, i.e. against atmospheric pressure.

Containers within the meaning of the invention can be e.g. flasks, pouches, bags or tubes. They can consist e.g. of glass, natural products or plastics. Containers made of glass are preferred, since these containers are odourless and permit very good cleaning. It is useful if a glass container has a moveable piston to enable it to be filled with breath.

Containers made of plastics, such as e.g. polyester (PET), polyethylene, polypropylene, polycarbonates and/or polyvinyl chloride and mixtures thereof, are particularly preferred. Pouches, bags or tubes are particularly advantageous as containers. These containers can be filled with a breath sample by normal exhalation, i.e. against atmospheric pressure. It is also preferred if the containers have a wall thickness of 1 μm to 500 μm. It is particularly easy to breathe into containers with this wall thickness without having to work noticeably harder than for normal exhalation against atmospheric pressure.

The containers can be either thrown away after use or cleaned using suitable cleaning agents.

The container usefully possesses an internal surface made of an odourless material. On the one hand, this prevents odour compounds from the container passing into the gas phase of the breath sample and biasing the result of the evaluation. On the other hand, it prevents odour-active components from precipitating on the internal wall of the container and no longer being available for the evaluation. Containers with an internal wall consisting of one or more plastics in which low adsorption of volatile organic components of the breath occurs are particularly preferred. In particular, therefore, pouches made of PET or with an internal PET coating are preferred.

It is also preferred if the container is filled with 20 ml to 7 l of breath in step a). Thus, the container can have the volume of a normal exhalation. However, it can also accept several exhalations, in this case having a correspondingly larger volume of up to 7 l, preferably 1 l to 5 1. In this case, the breath sample is a mixed sample of several exhalations. This advantageously facilitates the determination of the average nature and average strength of the odour, so that the reproducibility and objectivity of the evaluation are further improved.

In addition, it is advantageous to reduce further the microbial load of the breath sample contained in the container before evaluating the breath sample. To minimise the microbiological load, a filter can be used. This filter can be used in step a) to fill the container only with an already filtered breath sample. Alternatively or in addition, a filter can be provided in step c), so that the breath sample is forced through a filter for evaluation. In both cases, it must be ensured that the air stream is still sufficiently great. In preliminary tests, it has proved favourable if the air stream has a velocity of more than 30 ml/min and preferably no more than 500 ml/min.

The microorganisms in the container can optionally be killed before the evaluation by sterilisation, e.g. by strong UV radiation or radioactive radiation. In this case, it must be ensured that the sterilisation does not alter the intensity or nature of the sample odour. In particular, the sterilising process must not induce the container to give off odour-active substances into the gas phase of the breath sample or to remove them from the gas phase.

It is therefore particularly preferred to reduce the microbial load by condensation of moisture from the breath sample in the container. It has surprisingly been shown that, even with slight cooling of the container filled in step a), marked condensation of moisture from the breath takes place, a considerable proportion of the microbial load being removed from the gas phase of the breath sample and presumably being precipitated on the internal walls of the container. From there, the microbes do not re-enter the gas phase, or do so only to an insignificant extent. A particular advantage of this embodiment of the method is that the odour of the breath sample, i.e. the strength and nature of the odour thereof, are not changed by the reduction in the microbial load. Adequate and effective reduction of the microbial load of the breath sample is therefore possible just with the use of simple apparatus. It is particularly preferred if the container is adjusted to a temperature of 20° C. to 30° C. for the condensation. This temperature can usually be established even during a breath evaluation in summer.

It is also preferred to adjust the temperature of the breath sample to 20° C. to 40° C. before performing step c). The temperature of a normal breath sample lies within this range, and so the odour properties of breath can best be evaluated in this temperature range. The breath samples are preferably stored at room temperature before the evaluation.

The test subject will usefully fill the container with a breath sample in step a) by exhaling through the mouth, and preferably after inhaling through the nose. In this way, the mouth odour can be evaluated in a particularly unbiased manner.

The method according to the invention also makes it possible to evaluate one's own breath. To this end, the test subjects can fill the container with their own breath, usefully inhaling through the nose and exhaling through the mouth. The test subjects then evaluate their own breath from the container.

It is also preferred to anonymise the breath sample before the evaluation. For this purpose, it is particularly useful if the tester and test subject are not in the same room while step a) is being performed. Anonymisation means that the objectivity and reproducibility of an evaluation are improved particularly effectively.

To evaluate the breath from the container, the tester can squeeze the breath out of the container manually. It is preferred if the breath is squeezed out of the container by a suitable device. In this case, it is preferred if the breath is squeezed out of the container uniformly and reproducibly by the device.

The breath is usefully evaluated by a trained group of testers. The strength of the bad breath and of any flavouring present is usefully evaluated on a scale of 0 (no bad breath or no flavouring) to 9 (extremely bad breath or very high flavouring). It is particularly advantageous to use a large number of test subjects and/or a large group of testers in the evaluation. Both measures result in a clear improvement in the statistical significance of the evaluation results in each case.

The nature of the bad breath and of any flavouring present are usefully described by terms such as e.g. sulfurous, foul, faecal and protein. Any flavouring present is usefully described by the terms used in the production of flavourings, such as e.g. minty, fresh, aromatic and tangy.

Oral care products can preferably be evaluated as follows by the method according to the invention:
a) taking a first breath sample from a test subject using a method according to the invention as described above,
b) subsequently administering the oral care product to the test subject,
c) taking an additional breath sample from the test subject using a method in accordance with a method according to the invention as described above, at a pre-selected time after administering the oral care product, and
d) comparison of the first and the additional breath sample by a tester.

Oral care products within the meaning of the invention are agents used to improve the odour of breath. They can also be provided with specific preventative and curative properties and therefore, in addition to improving the odour of the breath, at the same time pursue therapeutic aims, e.g. the prevention of calculus formation, inflammations, bacterial attack, caries and periodontosis. Oral care products include, for example, flavoured and non-flavoured chewing gum, toothpaste, mouthwashes with alcohol, mouthwashes without alcohol, dental floss, oral cream and fixative cream. Dental treatments within the meaning of the invention are e.g. the removal of deposits in the oral cavity with the aim of improving the technical condition of the teeth and the breath.

In this evaluation method, the advantages described above for the method according to the invention can be exploited particularly effectively. To perform step d), the breath samples taken in steps a) and c) can be directly compared with one another by sniffing them. This is especially advantageous if less than an hour has passed between steps a) and d), preferably half an hour or less. Within this time, the odour of the breath sample taken in step a) has not yet changed significantly but can be compared directly with the fresh breath sample taken in step c). However, the breath sample taken in step a) can also be evaluated as described above before performing step c). In this case, the results of the evaluations of the breath samples taken in steps a) and c) are compared with one another in step d).

The invention is described in more detail below by means of the examples.

### EXAMPLE 1

### Comparison of the Type and Strength of Odour of Direct Breath With Indirect Breath From a Container

Ten test subjects each fill a coded PET pouch with approx. three litres of breath. The filling takes place by slow and even exhalation into the pouch through a nozzle. A tester then compares the direct breath from the test subjects with the indirect breath from the coded pouches with the aim of allocating the test subjects to the associated coded pouch.

It was found during this test that both the strength and the nature of the odour were identical, and so the allocation of persons to pouches was successful in every case.

In general, the filled pouches were to be kept at temperatures of 20° C. to 40° C. to avoid excessive alteration of the intensity and nature of the odour.

### EXAMPLE 2

### Evaluation of the General Recurring Mouth Odour in the Morning After Cleaning Teeth With a Flavoured Toothpaste

Conditioning of testers and test subjects: the test subjects are instructed to refrain from eating any strong-smelling foods such as garlic or similar for two days prior to the evaluation. In addition, the excessive consumption of alcohol and tobacco is forbidden. The test subjects are instructed not to clean their teeth after breakfast in the morning. The testers are instructed to use a flavour-free toothpaste on the day of the evaluation.

At the beginning of the evaluation, the test subjects each fill a coded PET pouch with approx. six litres of breath by slow, even exhalation through a nozzle. During this operation, the test subjects breathe in through the nose.

These pouches are then evaluated by the testers with respect to the typical mouth odour on a scale of 0 (no bad breath) to 9 (extremely bad breath) and this value is noted down as the current mouth odour intensity as the “zero value before teeth cleaning”.

The test subjects are then allocated to the individual test groups/test products by the trial leader. This allocation is made purely at random. Alternatively, the test subjects can also be allocated to the individual groups on the basis of the current mouth odour intensity. This enables groups to be formed with approximately the same average mouth odour intensity.

The test subjects then clean their teeth for three minutes using one gram of the relevant toothpaste.

Subsequently, each test subject fills a PET pouch with approx. five litres of breath each time, as just described, immediately and then every 30 minutes for a total of three hours.

These pouches are again evaluated anonymously by the testers within ten minutes. As before, the intensity of the typical mouth odour is evaluated. In addition, the testers also evaluate the intensity of the flavouring on a scale of 0 (no flavouring) to 9 (very strong flavouring). All the data are evaluated statistically. (Table 1)

Three toothpastes were prepared with the same basic formulation using the different flavourings KA21, BT97 and FJ46. Each of the three different toothpastes was then used by four test subjects to clean their teeth. The sensory evaluation of the test pouches of KA21, BT97 and FJ46 was performed in random order by three testers. The pouches were evaluated at the following times: 1) before cleaning the teeth, 2) 0 minutes after cleaning the teeth, 3) 30 minutes after cleaning the teeth, 4) 60 minutes after cleaning the teeth, 5) 90 minutes after cleaning the teeth, 6) 120 minutes after cleaning the teeth, 7) 150 minutes after cleaning the teeth and 8) 180 minutes after cleaning the teeth.

Each tester secretly notes a value for the typical mouth odour and a value for the strength of the flavouring.

TABLE 1
Average values of the test results of the three testers

| | Mouth odour | | | Flavouring | | |
|---|---|---|---|---|---|---|
| | KA21 | BT97 | FJ46 | KA21 | BT97 | FJ46 |
| beforehand | 6.8 | 6.7 | 6.8 | 0 | 0 | 0 |
| 0 min | 0.7 | 0.0 | 0.0 | 7.5 | 7.7 | 7.4 |
| 30 min | 1.4 | 0.6 | 0.5 | 6.3 | 6.0 | 5.9 |
| 60 min | 1.9 | 1.4 | 1.4 | 5.0 | 5.2 | 5.3 |
| 90 min | 2.8 | 2.3 | 2.1 | 3.9 | 3.9 | 4.1 |
| 120 min | 4.5 | 3.3 | 3.0 | 2.0 | 2.1 | 2.3 |
| 150 min | 6.3 | 4.1 | 3.5 | 0.5 | 0.3 | 0.6 |
| 180 min | 7.0 | 5.4 | 4.7 | 0 | 0 | 0 |

The flavoured toothpastes BT97 and FJ46 display a markedly better and more long-lasting reduction in mouth odour compared with KA21. Thus, the toothpaste KA21 has a value of 7 at 180 minutes while the toothpastes BT97 and FJ46 are at least 1.5 units lower. The flavouring intensities differ only slightly.

It is possible, with the aid of the method according to the invention, to evaluate the different properties of the flavourings KA21, BT97 and FJ46 in relation to the reduction of typical mouth odour.

### EXAMPLE 3

### Comparison of the Masking of Mouth Odour After Garlic by Two Mouthwashes Before and After Use

Conditioning of testers and test subjects: the test subjects are instructed to refrain from eating any strong-smelling foods such as garlic or similar for two days prior to the evaluation. In addition, the excessive consumption of alcohol and tobacco is forbidden. The testers are instructed to use a flavour-free toothpaste on the day of the evaluation.

At the beginning of the evaluation, the test subjects are given 50 g of a food seasoned with garlic to eat. After 10 minutes, the test subjects each fill a coded PET pouch with approx. six litres of breath by slow, uniform exhalation through a nozzle. During this operation, the test subjects breathe in through the nose.

These pouches are then evaluated by the testers with respect to the typical garlic odour on a scale of 0 (no garlic) to 9 (extremely strong garlic). The twelve test subjects are then allocated to the two mouthwashes by the trial leader with the aim of forming two groups with approximately the same average garlic intensity.

Each of the two mouthwashes is then used by six test subjects for 15 seconds. Subsequently, the test subjects fill further coded pouches immediately and after 60 minutes, and each of these is evaluated by the four testers. Each tester secretly notes a value for the typical garlic odour and a value for the strength of the flavouring.

TABLE 2
Average values of the test results

| | |
|---|---|
| Garlic odour | Flavouring |

| | PB72 | AJ46 | PB72 | AJ46 |
|---|---|---|---|---|
| beforehand | 8.3 | 8.2 | 0 | 0 |
| 0 min | 1.5 | 3.9 | 7.8 | 6.4 |
| 60 min | 2.9 | 4.8 | 5.9 | 4.1 |

The mouthwash PB72 is clearly better able to reduce the odour of garlic and also possesses a distinctly more intense flavouring.

It is possible, with the aid of the new method, to evaluate the properties of mouthwashes and to identify products that are more effective.

1. A method of evaluating the breath of a test subject, comprising the following steps:
a) collection of a breath sample from the test subject in a container,
b) reduction of the microbial load of the breath sample contained in the container, and then
c) evaluation by a tester of the breath sample collected in the container.

2. A method according to claim 1, characterized in that, in step a), the container is filled with breath by the test subject against atmospheric pressure by exhalation.

3. A method according to claim 1 characterized in that the volume of the container is variable, the container preferably being collapsible.

4. A method according to claim 1 characterized in that the container possesses an internal surface made of an odourless material.

5. A method according to claim 1 characterized in that the container is filled with 20 ml to 7 1 of breath in step a).

6. A method according to claim 1 characterized in that the microbial load is reduced by condensation of moisture from the breath sample in the container.

7. A method according to claim 1 characterized in that the temperature of the breath sample is adjusted to a value of 20° C. to 40° C. before performing step c).

8. A method according to claim 1 characterized in that the test subject fills the container in step a) by exhaling through the mouth, and preferably after inhaling through the nose.

9. A method according to claim 1 characterized in that the breath sample is anonymised prior to evaluation.

10. A method of evaluating an oral care product, comprising the following steps:
a) taking a first breath sample from a test subject by a method according to claim 1,
b) subsequently administering the oral care product to the test subject,
c) taking an additional breath sample from the test subject using a method according to claim 1 at a pre-selected time after administering the oral care product, and
d) comparing the first and the additional breath samples by a tester.
